(19) United States
(12) Patent Application Publication
(10) Pub. No.: US 2005/0131239 A1
(43) Pub. Date: Jun. 16, 2005

(54) Preparation of aryl-substituted butenolides using mucohalic acids

(76) Inventors: Peter Garth Blazecka, Windsor (CA); Ji Zhang, Ann Arbor, MI (US)

Correspondence Address: WARNER-LAMBERT COMPANY, 2800 PLYMOUTH RD, ANN ARBOR, MI, 48105, US

(21) Appl. No.: 10/987,531

(22) Filed: Nov. 12, 2004

Related U.S. Application Data
(60) Provisional application No. 60/520,233, filed on Nov. 14, 2003.

(51) Int. Cl. C07D 307/02

(52) U.S. Cl. 549/295

(57) ABSTRACT

Methods and materials for preparing 3-aryl-2-buten-4-olides and 2,3-bisaryl-2-buten-4-olides are disclosed. The methods include reacting a mucohalic acid with a reducing agent to give a 2,3-dihalo-2-buten-4-olide, which undergoes at least one Pd catalyzed cross-coupling reaction with an arylboronic acid.

### CROSS REFERENCE TO RELATED APPLICATION

This application claims the benefit of U.S. Provisional Application No. 60/520,233, filed Nov. 14, 2003, the complete disclosure of which is herein incorporated by reference.

### BACKGROUND OF THE INVENTION

1. Field of Invention

This invention relates to materials and methods for preparing aryl mono- or bis-substituted butenolides, which are biologically active natural products and compounds having anti-viral, anti-inflammatory or anti-cancer activity.

2. Discussion

Substituted butenolides have attracted considerable attention due to their interesting biological properties. Useful substituted butenolides include 2,3-bisaryl-2-buten-4-olides, including rubrolides I, K, L, and M, which are potentially useful anti-cancer agents (F. Bellina et al., Tetrahedron Lett. (2002) 43:2023; F. Bellina et al., Tetrahedron Lett. (2001) 42:3851; F. Bellina et al., Tetrahedron (2000) 57:9997). Other 2,3-bisaryl-2-buten-4-olides include 3-(4-methanesulfonylphenyl)-2-phenyl-2-buten-4-olide (rofecoxib), which is the active ingredient in the anti-inflammation drug VIOXX® (M. Therien et al., Synthesis (2001) 12:1778; P. Forgione et al., Tetrahedron Lett. (2000) 41:17). Other useful substituted butenolides include 3-monoaryl-2-buten-4-olides, such as succinic acid mono-{1-[3-methyl-5-(5-oxo-2,5-dihydro-furan-3-yl)-benzofuran-2-yl]-ethyl}ester, which is the active ingredient in the vasodilator EUCILAT® (J. Schmit et al., Chim. Ther. (1966) 5-6:305; J. Schmitt et al., Bull. Soc. Chim. Fr. (1967) 1:74; J. Vallat et al., Eur. Med. Chem. (1981) 16:409).

Although researchers have developed techniques for preparing substituted butenolides, these methods can be improved. Many of the techniques use expensive starting materials, potentially toxic reagents, or harsh reactions conditions, which make them impractical for commercial production. Thus, new methods for preparing substituted butenolides are needed.

### SUMMARY OF THE INVENTION

The present invention provides methods for preparing 3-aryl-2-buten-4-olides, and 2,3-bisaryl-2-buten-4-olides. When compared with existing methods, the claimed methods employ mild reaction conditions, and use comparatively inexpensive, and benign starting materials and reagents.

Therefore, one aspect of the present invention provides a method of making a compound of Formula 1, 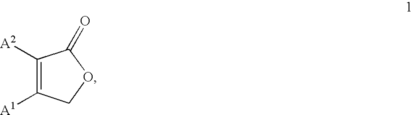
wherein Ar¹ and Ar² are the same or different, and are aryl. The method comprises: reacting a compound of Formula 2, 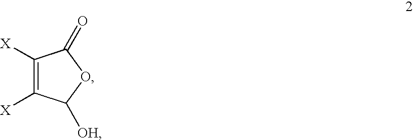
with a reducing agent, in the presence of an acid catalyst and a first solvent to yield a compound of Formula 3, 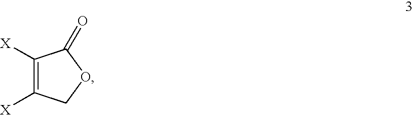
wherein X in Formula 2 and in Formula 3 is halogen;
(b) reacting the compound of Formula 3 with a compound of Formula 4,
   Ar¹—B(OH)₂ 4,
   at temperature T₁, and in the presence of a first base, a first Pd catalyst, a first phase transfer catalyst, a second solvent, and H₂O, to yield the compound of Formula 5, 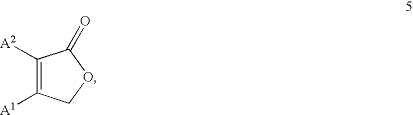
   wherein Ar¹ in Formula 4 and Formula 5 are as defined above for Formula 1, X is as defined above for Formula 2 and Formula 3, and the second solvent is the same as or different than the first solvent; and
(c) reacting the compound of Formula 5 with a compound of Formula 6,
   Ar²—B(OH)₂ 6,
   at temperature T₂, and in the presence of a second base, a second Pd catalyst, a second phase transfer catalyst, a third solvent, and H₂0, to yield the compound of Formula 1;
wherein Ar² in Formula 6 is as defined above in Formula 1;
the second base, the second Pd catalyst, the second phase transfer catalyst, and the third solvent are the same as or different than, the first base, the first Pd catalyst, the first phase transfer catalyst, and the second solvent, respectively; and
T₂ is greater than T₁.

Another aspect of the present invention provides a method of making a compound of Formula 7, 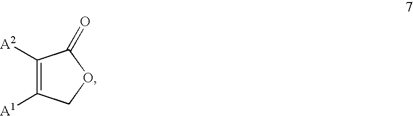
wherein Ar¹ is aryl. The method comprises:
(a) reacting a compound of Formula 2, 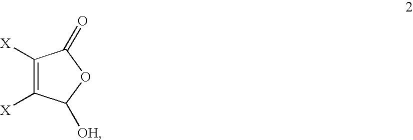
   with a reducing agent, in the presence of an acid catalyst and a first solvent to yield a compound of Formula 3, 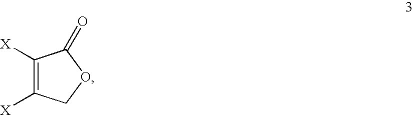
   wherein X in Formula 2 and Formula 3 is halogen;
(b) reacting the compound of Formula 3 with a compound of Formula 4,
   Ar¹—B(OH)₂ 4,
   at a temperature above room temperature, and in the presence of a base, a Pd catalyst, a phase transfer catalyst, a second solvent, and H₂O, to yield the compound of Formula 7;
wherein Ar¹ in Formula 4 is as defined above for Formula 7, X is as defined above for Formula 2 and Formula 3, and the second solvent is the same as or different than the first solvent.

A further aspect of the present invention provides a method of making a compound of Formula 5, 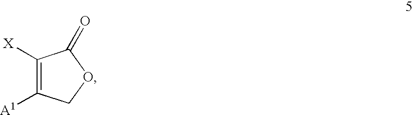
wherein Ar¹ is aryl, and X is halogen. The method comprises:
(a) reacting a compound of Formula 2, 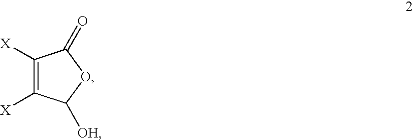
   with a reducing agent, in the presence of an acid catalyst and a first solvent to yield a compound of Formula 3, 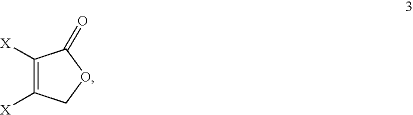
   wherein X in Formula 2 and in Formula 3 is as defined above in Formula 5;
(b) reacting the compound of Formula 3 with a compound of Formula 4,
   Ar¹—B(OH)₂ 4,
   at a temperature below reflux temperature, and in the presence of a base, a Pd catalyst, a phase transfer catalyst, a second solvent, and H₂O, to yield the compound of Formula 5;
wherein Ar¹ in Formula 4 is as defined above for Formula 5, and the second solvent is the same as or different than the first solvent.

### DETAILED DESCRIPTION

Definitions and Abbreviations

Unless otherwise indicated, this disclosure uses definitions provided below. Some of the definitions and formulae may include a “—” (dash) to indicate a bond between atoms or a point of attachment to a named or unnamed atom or group of atoms. Other definitions and formulae may include an “═” to indicate a double bond.

“Substituted” groups are those in which one or more hydrogen atoms have been replaced with one or more non-hydrogen groups, provided that valence requirements are met and that a chemically stable compound results from the substitution.

“Alkyl” refers to straight chain and branched saturated hydrocarbon groups, generally having a specified number of carbon atoms (i.e., C₁₋₆ alkyl refers to an alkyl group having 1, 2, 3, 4, 5, or 6 carbon atoms. Examples of alkyl groups include, without limitation, methyl, ethyl, n-propyl, i-propyl, n-butyl, s-butyl, i-butyl, t-butyl, pent-1-yl, pent-2-yl, pent-3-yl, 3-methylbut-1-yl, 3-methylbut-2-yl, 2-methylbut-2-yl, 2,2,2-trimethyleth- 1-yl, n-hexyl, and the like.

“Alkenyl” refers to straight chain and branched hydrocarbon groups having one or more unsaturated carbon-carbon bonds, and generally having a specified number of carbon atoms. Examples of alkenyl groups include, without limitation, ethenyl, 1-propen-1-yl, 1-propen-2-yl, 2-propen-1-yl, 1-buten-1-yl, 1-buten-2-yl, 3-buten-1-yl, 3-buten-2-yl, 2-buten-1-yl, 2-buten-2-yl, 2-methyl-1-propen-1-yl, 2-methyl-2-propen-1-yl, 1,3-butadien-1-yl, 1,3-butadien-2-yl, and the like.

“Alkynyl” refers to straight chain or branched hydrocarbon groups having one or more triple carbon-carbon bonds, and generally having a specified number of carbon atoms. Examples of alkynyl groups include, without limitation, ethynyl, 1-propyn-1-yl, 2-propyn-1-yl, 1-butyn-1-yl, 3-butyn-1-yl, 3-butyn-2-yl, 2-butyn-1-yl, and the like.

“Alkanoyl” refers to alkyl-C(O)—, where alkyl is defined above, and generally includes a specified number of carbon atoms, including the carbonyl carbon. Examples of alkanoyl groups include, without limitation, formyl, acetyl, propionyl, butyryl, pentanoyl, hexanoyl, and the like.

“Alkoxy,” “alkoxycarbonyl,” and “alkoxycarbonyloxy” refer, respectively, to alkyl-O—, alkyl-O—C(O)—, and alkyl-O—C(O)—O—, where alkyl is defined above. Examples of alkoxy groups include, without limitation, methoxy, ethoxy, n-propoxy, i-propoxy, n-butoxy, s-butoxy, t-butoxy, n-pentoxy, s-pentoxy, and the like.

“Cycloalkyl” refers to saturated monocyclic and bicyclic hydrocarbon rings, generally having a specified number of carbon atoms that comprise the ring (i.e., C₃₋₇ cycloalkyl refers to a cycloalkyl group having 3, 4, 5, 6 or 7 carbon atoms as ring members and C₃₋₁₂ cycloalkyl refers to a cycloalkyl group having 3, 4, 5, 6, 7, 8, 9, 10, 11, or 12 carbon atoms as ring members). Cycloalkyl groups may be attached to a parent group or to a substrate at any ring atom, unless such attachment would violate valence requirements. Likewise, the cycloalkyl group may include one or more non-hydrogen substituents unless such substitution would violate valence requirements. Useful substituents include, without limitation, alkyl, alkoxy, alkoxycarbonyl, and alkanoyl, as defined above, and hydroxy, mercapto, nitro, halogen, and amino.

Examples of monocyclic cycloalkyl groups include, without limitation, cyclopropyl, cyclobutyl, cyclopentyl, cyclohexyl, and the like. Examples of bicyclic cycloalkyl groups include, without limitation, bicyclo[1.1.0]butyl, bicyclo[1.1.1]pentyl, bicyclo[2.1.0]pentyl, bicyclo[2.1.1]hexyl, bicyclo[3.1.0]hexyl, bicyclo[2.2.1]heptyl, bicyclo[3.2.0]heptyl, bicyclo[3.1.1]heptyl, bicyclo[4.1.0]heptyl, bicyclo[2.2.2]octyl, bicyclo[3.2.1]octyl, bicyclo[4.1.1]octyl, bicyclo[3.3.0]octyl, bicyclo[4.2.0]octyl, bicyclo[3.3.1]nonyl, bicyclo[4.2.1]nonyl, bicyclo[4.3.0]nonyl, bicyclo[3.3.2]decyl, bicyclo[4.2.2]decyl, bicyclo[4.3.1]decyl, bicyclo[4.4.0]decyl, bicyclo[3.3.3]undecyl, bicyclo[4.3.2]undecyl, bicyclo[4.3.3]dodecyl, and the like, which may be attached to a parent group or substrate at any of the ring atoms, unless such attachment would violate valence requirements.

“Cycloalkanoyl” refers to cycloalkyl-C(O)—, where cycloalkyl is defined above, and generally includes a specified number of carbon atoms, excluding the carbonyl carbon. Examples of cycloalkanoyl groups include, without limitation, cyclopropanoyl, cyclobutanoyl, cyclopentanoyl, cyclohexanoyl, cycloheptanoyl, and the like.

“Halo,” “halogen” and “halogeno” may be used interchangeably, and refer to fluoro, chloro, bromo, and iodo.

“Haloalkyl” and “haloalkanoyl” refer, respectively, to alkyl or alkanoyl groups substituted with one or more halogen atoms, where alkyl and alkanoyl are defined above. Examples of haloalkyl and haloalkanoyl groups include, without limitation, trifluoromethyl, trichloromethyl, pentafluoroethyl, pentachloroethyl, trifluoroacetyl, trichloroacetyl, pentafluoropropionyl, pentachloropropionyl, and the like.

“Heterocycle” and “heterocyclyl” refer to 5- to 7-membered monocyclic or bicyclic rings or to 7- to 10-membered bicyclic rings, which are saturated, partially unsaturated, or unsaturated. These groups have ring members made up of carbon atoms and from 1 to 4 heteroatoms that are independently nitrogen, oxygen or sulfur, and may include any bicyclic group in which any of the above-defined heterocycles are fused to a benzene ring. The nitrogen and sulfur heteroatoms may optionally be oxidized. The heterocyclic ring may be attached to a parent group or substrate at any heteroatom or carbon atom, unless such attachment would violate valence requirements. Likewise, the heterocyclyl groups may be substituted on a carbon or on a nitrogen atom, unless such substitution would violate valence requirements. Useful substituents include, without limitation, alkyl, alkoxy, alkoxycarbonyl, alkanoyl, and cycloalkanoyl, as defined above, and hydroxy, mercapto, nitro, halogen, and amino.

Examples of heterocycles include, without limitation, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H, 6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazotyl, 1,2,5-triazolyl, 1,3,4-triazolyl, and xanthenyl.

“Aryl” refers to aromatic groups, including heterocyclic groups as defined above, which are aromatic. Examples of aryl groups include, without limitation, phenyl, naphthyl, biphenyl, pyrenyl, anthracenyl, fluorenyl, pyrimidinyl, purinyl, imidazolyl, indolyl, quinolinyl isoquinolinyl, and the like, which may be unsubstituted or substituted with 1 to 4 substituents such as alkyl, alkoxy, alkoxycarbonyl, alkanoyl, and cycloalkanoyl, as defined above, and hydroxy, mercapto, oxy, nitro, halogen, and amino.

“Arylalkyl” refers to aryl-alkyl, where aryl and alkyl are defined above. Examples include, without limitation, benzyl, fluorenylmethyl, and the like.

Table 1 lists abbreviations, which are used throughout the specification.

TABLE 1
List of Abbreviations

| Abbreviation | Description |
|---|---|
| Ac | acetyl |
| ACN | acetonitrile |
| Aq | aqueous |
| Bn | benzyl |
| Bu | butyl |
| t-Bu | tertiary butyl |
| CO₂Me | methoxycarbonyloxy |
| CO₂t-Bu | tertiarybutoxycarbonyloxy |
| COMe | methylcarbonyl (acetyl) |
| d | day |
| DMF | dimethylformamide |
| DMSO | dimethylsulfoxide |
| Et | ethyl |
| ET₃N | triethylamine |
| EtOH | ethyl alcohol |
| Et₂O | ethyl ether |
| EtOAc | ethyl acetate |
| h | hour |
| Me | methyl |
| MeOH | methyl alcohol |
| min | minute |
| NaOAc | sodium acetate |
| NH₄OAc | ammonium acetate |
| NMP | N-methylpyrrolidone |
| NR | no reaction |
| 3-OCH₂O-4 | methylenedioxy |
| p-OMe | para-methoxy |
| PdCl₂(dppf)₂ | dichloro[1,1′-bis(diphenylphosphino)ferrocene]palladium |
| | (II) dichloromethane adduct |
| Pd₂(dba)₃ | tris(dibenzylidene-acetone)dipalladium(0) |
| Pd(PPh₃)₄ | tetrakis(triphenylphosphine)palladium(0) |
| Ph | phenyl |
| Ph₃P | triphenylphosphine |
| Pr | propyl |
| ppm | parts per million |
| i-Pr | isopropyl |
| i-PrOH | isopropyl alcohol |
| RT | room temperature (approximately 20° C. to 25° C.) |
| TFA | trifluoroacetic acid |
| THF | tetrahydrofuran |
| Ti(Oi-Pr)₄ | titanium tetraisopropoxide |
| TLC | thin-layer chromatography |

In some of the reaction schemes and examples below, certain compounds can be prepared using protecting groups, which prevent undesirable chemical reaction at otherwise reactive sites. Protecting groups may also be used to enhance solubility or otherwise modify physical properties of a compound. For a discussion of protecting group strategies, materials and methods for installing and removing protecting groups, and a compilation of useful protecting groups for common functional groups, including amines, carboxylic acids, alcohols, ketones, aldehydes, and the like, see T. W. Greene and P. G. Wuts, Protecting Groups in Organic Chemistry (1999) and P. Kocienski, Protective Groups (2000), which are herein incorporated by reference in their entirety for all purposes.

In addition, some of the schemes and examples below may omit details of common reactions, including oxidations, reductions, and so on, which are known to persons of ordinary skill in the art of organic chemistry. The details of such reactions can be found in a number of treatises, including Richard Larock, Comprehensive Organic Transformations (1999), and the multi-volume series edited by Michael B. Smith and others, Compendium of Organic Synthetic Methods (1974-2003). Starting materials and reagents may be obtained from commercial sources or may be prepared from literature sources.

Generally, the chemical transformations described throughout the specification may be carried out using substantially stoichiometric amounts of reactants, though certain reactions may benefit from using an excess of one or more of the reactants. Additionally, some of the reactions disclosed in the specification may be carried out at about RT, but some reactions may benefit from the use of higher or lower temperatures, depending on reaction kinetics, yields, and the like.

Many of the chemical transformations may employ one or more compatible solvents, which depending on the nature of the reactants, may be polar protic solvents, polar aprotic solvents, non-polar solvents, or some combination. Although the choice of solvent or solvents may influence the reaction rate and yield, such solvents are generally considered to be inert (unreactive).

Scheme I shows a method of making 2,3-bisaryl-2-buten-4-olides of Formula 1. The method includes reacting a mucohalic acid of Formula 2 with a reducing agent in the presence of a solvent and acid catalyst to give a 2,3-dihalo-2-buten-4-olide of Formula 3. The compound of Formula 3 is reacted with an arylboronic acid of Formula 4, which yields the 3-aryl-2-buten-4-olide of Formula 5, which is subsequently reacted with an arylboronic acid of Formula 6 to yield the 2,3-bisaryl-2-buten-4-olide of Formula 1. In Formula 1 to Formula 6, Ar¹ and Ar² may be the same or different, and are aryl, and X is halogen (Cl, Br, or I). Useful X include Cl and Br, and useful Ar¹ and Ar² include phenyl and indolyl, each optionally having one or more non-hydrogen substituents, including C₁₋₆ alkyl, C₁₋₆ haloalkyl, C₁₋₆ alkoxy, or halogen.

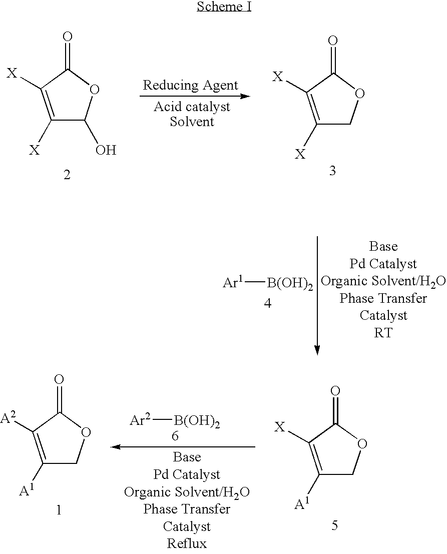

Although conversion of the mucohalic acids of Formula 2 to the 2,3-dihalo-2-buten-4-olides of Formula 3 depends somewhat on the choice of reagents, a wide variety of reducing agents, acid catalysts, and solvents may be used. Useful reducing agents include, without limitation, sodium triacetoxyborohydride, sodium cyanoborohydride, triethyl silane, Ti(Oi-Pr)₄/NaBH₃CN, borohydride exchange resin, Zn/acetic acid, sodium borohydride/magnesium perchlorate, and zinc borohydride/zinc chloride. Useful acid catalysts include, without limitation, protic acids, such as acetic acid, trichloroacetic acid, trifluoroacetic acid, formic acid, and the like, as well as non-protic acids, such as magnesium chloride, magnesium triflate, boron trifluoride etherate, AlCl₃, FeCl₃, ZnCl₂, AlBr₃, ZnB₂, TiCl₄, SiCl₄, SnCl₄, and the like. Useful solvents include, without limitation, aprotic solvents, such as 1,4-dioxane, THF, Et₂O, dichloromethane, trichloromethane, dichloroethane, nitromethane, ACN, NMP, DMF, DMSO, and the like.

The conversion of the mucohalic acids of Formula 2 to the 2,3-dihalo-2-buten-4-olides of Formula 3 can be undertaken using substantially stoichiometric amounts of reactants, though it may be advantageous to carryout the reaction with an excess of the reducing agent. For example, the molar ratio of the reducing agent to mucohalic acid may range from about 1 to about 5, but more typically ranges from about 1.5 to about 3, and usually ranges from about 1.5 to about 2.0. The amount of acid catalyst used is sufficient to maintain a pH of about 2 to about 7. Generally, any reference in the disclosure to a stoichiometric range, a temperature range, a pH range, etc., includes the indicated endpoints.

As shown in the examples below, the reduction of mucohalic acids at room temperature (RT) results in good yields of 2,3-dihalo-2-buten-4-olides. Moreover, all of the conversions occur within a reasonable period of time (i.e., reaction times under about 72 hours). To modify reaction time and yield, however, the reaction temperature may be varied from about −10° C. to about 60° C.

The 3-aryl-2-buten-4-olides of Formula 5, and the 2,3-bisaryl-2-buten-4-olides of Formula 1 are obtained through Pd-catalyzed cross-coupling reactions (Suzuki couplings) with arylboronic acids (Formula 4 and Formula 6). Both cross-couplings utilize phase-transfer methodology, which appears to favor the desired palladium insertion in the organic layer, while minimizing unwanted side reactions in the aqueous layer. Useful phase transfer catalysts include, but are not limited to, tetraalkylammonium salts, benzyltrialkylammonium salts, tetralkylphosphonium salts, crown ethers, and polyethylene glycols. Particularly useful phase transfer catalysts include benzyltrialkylammonium salts, such as BnEt₃N⁺Cl⁻.

The cross-couplings also employ a base, Pd catalyst, and organic solvent. Useful bases include, without limitation, non-aqueous bases (e.g., CsF and KF), inorganic bases, such as Group 1 metal carbonates (e.g., Li₂CO₃, Na₂CO₃, NaHCO₃, K₂CO₃, and Cs₂CO₃) and phosphates (e.g., K₂HPO₄ and K₃PO₄), and other bases that are soluble in the organic solvent, such as NaOAc, NH₄OAc, alkyl₄NF, and the like. Useful Pd catalysts include, without limitation, Pd(Ph₃)₄, PdCl₂(Ph₃)₂, etc., and suitable solvents include, without limitation, aromatic solvents, such as toluene, 1,2-dichlorobenzene, 1,2,4-trichlorobenzene, pentafluorobenzene, trifluorotoluene, and the like, as well as those solvents listed above with respect to the reduction of mucohalic acid.

Generally, the cross-coupling reactions employ excess base (e.g., from about 2 equiv. to about 6 equiv.) and excess arylboronic acid (e.g., from about 2 equiv. to about 4 equiv., and more typically from about 2 equiv. to about 3 equiv.). The amount of Pd catalyst and phrase transfer catalyst used may independently range from about 1 mol % to about 20 mol %, typically ranges from about 1 mol % to about 15 mol %, and often ranges from about 5 mol % to about 10 mol %.

As can be seen in Scheme I, the two cross-couplings differ in reaction temperature. The first cross-coupling reaction occurs at about RT or below, and as shown in the examples, generates the 3-aryl-2-buten-4-olide from mucochloric acid or mucobromic acid in good yields (91% to 99% and 78% to 91%, respectively) in about 48 to 72 h. Surprisingly, no regioisomer (i.e., 2-aryl-2-buten-4-olide) is detected. The second cross-coupling reaction occurs at about reflux temperatures, and generates the 2,3-bisaryl-2-buten-4-olide from mucochloric acid or mucobromic acid in good yields (71% to 99% and 41% to 88%, respectively).

As noted in the examples, when Ar¹ and Ar² are the same, the bisarylation of the mucohalic acid may be undertaken in a single step under reflux conditions. Similarly, as illustrated in Examples 20 to 25, which describe the preparation of 3-(4-methanesulfonylphenyl)-2-phenyl-2-buten-4-olide (rofecoxib) from 2,3-dibromo-2-buten-4-olide (Example 20, 22, and 25) or 2,3-dichloro-2-buten-4-olide (Example 21, 23, and 24), the 3-aryl-2-buten-4-olide (monoarylated butenolide) may undergo further chemical transformation before reaction with the second cross-coupling reaction.

Some of the compounds disclosed in this specification may contain one or more asymmetric carbon atoms and therefore may exist as optically active stereoisomers (i.e., pairs of enantiomers). Some of the compounds may also contain an alkenyl or cyclic group, so that cis/trans (or Z/E) stereoisomers (i.e., pairs of diastereoisomers) are possible. Still other compounds may exist as one or more pairs of diastereoisomers in which each diastereoisomer exists as one or more pairs of enantiomers. Finally, some of the compounds may contain a keto or oxime group, so that tautomerism may occur. In such cases, the scope of the present invention includes individual stereoisomers of the disclosed compound, as well as its tautomeric forms (if appropriate).

Individual enantiomers may be prepared or isolated by known techniques, such as conversion of an appropriate optically-pure precursor, resolution of the racemate (or the racemate of a salt or derivative) using, for example, chiral HPLC, or fractional crystallization of diastereoisomeric salts formed by reaction of the racemate with a suitable optically active acid or base (e.g., tartaric acid). Diastereoisomers may be separated by known techniques, such as fractional crystallization and chromatography.

The disclosed compounds also include all isotopic variations, in which at least one atom is replaced by an atom having the same atomic number, but an atomic mass different from the atomic mass usually found in nature. Examples of isotopes suitable for inclusion in the disclosed compounds include, without limitation, isotopes of hydrogen, such as ²H and ³H; isotopes of carbon, such as ¹³C and ¹⁴C; isotopes of nitrogen, such as ¹⁵N; isotopes of oxygen, such as ¹⁷O and ¹⁸O; isotopes of phosphorus, such as ³¹P and ³²P; isotopes of sulfur, such as ³⁵S; isotopes of fluorine, such as ¹⁸F; and isotopes of chlorine, such as ³⁶Cl.

### EXAMPLES

The following examples are intended to be illustrative and non-limiting, and represent specific embodiments of the present invention.

General Methods

All reactions were carried out under nitrogen atmosphere unless otherwise noted. All solvents and reagents used were from commercial sources and no further purification was performed. Reactions were monitored by mass spectrometry (MS) on a MICROMASS PLATFORM LC and by thin-layer chromatography on 0.25 mm E. MERCK silica gel 60 plates (F₂₅₄) using UV light and aqueous potassium permanganate-sodium bicarbonate as visualizing agents. E. MERCK silica gel 60 (0.040 to 0.063 mm and 0.063 to 0.200 mm particle sizes) was used for column chromatography. Melting points were determined using a THOMAS-HOOVER melting point apparatus in open capillaries and were not corrected. Proton nuclear magnetic resonance (¹H NMR) spectra were recorded at 400 MHz on a VARIAN UNITY INOVA AS400. Chemical shifts are reported as delta (δ) units in parts per million (ppm) relative to the singlet at 7.26 ppm for deuteriochloroform. Coupling constants (J) are reported in Hertz (Hz). Carbon-13 nuclear magnetic resonance (¹³C NMR) spectra were recorded at 100 MHz on a VARIAN UNITY PLUS INOVA 400. Chemical shifts are reported as delta (δ) units in parts per million (ppm) relative to the center line of the triplet at 77.3 ppm for deuteriochloroform or the center line of the septet at 40.2 ppm for DMSO-d₆. Chemical shifts are reported as delta (δ) units in parts per million (ppm) relative to deuteriochloroform. Elemental analyses were performed out-of-house by QUANTITATIVE TECHNOLOGIES INC.

### Example 1

### Preparation of 2,3-dichloro-2-buten-4-olide

Mucochloric acid (20.0 g, 0.118 mol) and sodium triacetoxyborohydride (37.6 g, 0.178 mol) were suspended in chloroform (600 mL) and cooled to 0° C. to 5° C. Acetic acid (12 mL) was added and the reaction mixture was allowed to warm to 20° C. to 25° C. Stirring at 20° C. to 25° C. was continued for 3 d. The reaction mixture was diluted with water (100 mL) and the resulting aqueous and organic phases were separated. The organic phase was washed once with water (100 mL) and was concentrated under reduced pressure. The residue was purified by silica gel column chromatography (CH₂Cl₂) followed by recrystallization (CH₂Cl₂-heptane) to give 2,3-dichloro-2-buten-4-olide as a white solid: yield 8.08 g (45%), mp 46° C. to 47° C. ¹H NMR (CDCl₃): δ 4.87 (s, 2H). ¹³C NMR (CDCl₃): δ 166.1, 149.3, 121.2, 71.2. Anal. Calc'd for C₄H₂Cl₂O₂: C, 31.41; H, 1.32; Cl, 46.35. Found: C, 31.31; H, 1.17; Cl, 46.08.

### Example 2

### Preparation of 2,3-dibromo-2-buten-4-olide

Mucobromic acid (2, 20.0 g, 0.0776 mol) and sodium triacetoxyborohydride (24.7 g, 0.116 mol) were suspended in chloroform (600 mL) and cooled to 0° C. to 5° C. Acetic acid (12 mL) was added and the reaction mixture was allowed to warm to 20° C. to 25° C. Stirring at 20° C. to 25° C. was continued for 3 d. The reaction mixture was diluted with water (100 mL) and the resulting aqueous and organic phases were separated. The organic phase was washed once with water (100 mL) and was concentrated under reduced pressure. The residue was purified by silica gel column chromatography (CH₂Cl₂) followed by recrystallization (CH₂Cl₂-heptane) to give 2,3-dibromo-2-buten-4-olide as a white solid: yield 10.7 g (57%), mp 87° C. to 89° C. ¹H NMR (CDCl₃): δ 4.87 (s, 2H). ¹³C NMR (CDCl₃): δ 166.9, 143.9, 114.8, 74.4. Anal. Calc'd for C₄H₂Br₂O₂: C, 19.86; H, 0.83; Br, 66.07. Found: C, 19.94; H, 0.79; Br, 66.15.

### Examples 3-11

### Preparation of 2,3-diphenyl-2-buten-4-olide (Formula 8)

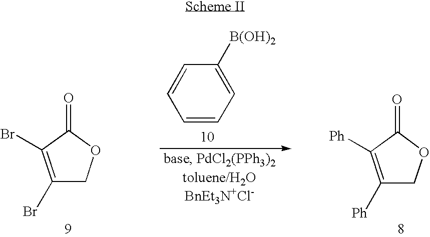

Table 1 lists bases, reaction times, and yields for various Suzuki couplings of 2,3-dibromo-2-buten -4-olide (Formula 9) and phenylboronic acid (Formula 10). For each of the entries, 2,3-dibromo-2-buten-4-olide (2.5 mmol) was combined with phenylboronic acid (2.4 equiv.), a desired base (4.0 equiv.), PdCl₂(PPh₃)₂ (5 mol %), BnEt₃N⁺Cl⁻ (5 mol %) and degassed toluene (10 mL) and water (10 mL). The reaction mixture was refluxed for 2 h to 3 h, and was subsequently partitioned between 2 N hydrochloric acid (10 mL) and toluene (100 mL). The toluene extract was concentrated under reduced pressure, and the resulting residue was purified by silica gel column chromatography to yield a pale yellow solid

TABLE 1
Preparation of 2,3-diphenyl-2-buten-4-olide (Formula 8) via coupling of
2,3-dibromo-2-buten-4-olide and phenylboronic acid

| Example | Base | Time^{a}, h | Yield^{b} |
|---|---|---|---|
| 3 | K₃PO₄ | 2 | 56 |
| 4 | K₂CO₃ | 3 | 42 |
| 5 | Cs₂CO₃ | 3 | 51 |
| 6 | Na₂CO₃ | 3 | 41 |
| 7 | NaHCO₃ | 3 | 68 |
| 8 | K₂HPO₄ | 3 | 68 |
| 9 | KF | 3 | 71 |
| 10 | CsF | 3 | 78 |
| 11 | CsF | 24 | 88 |

^{a}Reaction times were not optimized
^{b}Isolated yields after chromatography

### Example 12-17

### Preparation of 2,3-bisaryl-2-buten-4-olide (Formula 13)

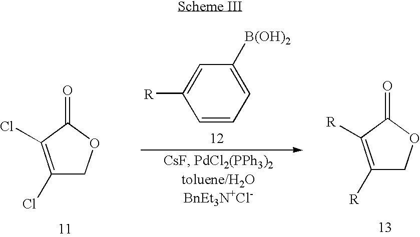

Table 2 lists yields for various Suzuki couplings of 2,3-dichloro-2-buten-4-olide (Formula 11) and various arylboronic acids (Formula 12). For each of the entries, 2,3-dichloro-2-buten-4-olide (1.8-2.5 mmol) was combined with an m-substituted phenylboronic acid (3.0 equiv.), cesium fluoride (4.0 equiv.), PdCl₂(PPh₃)₂ (5 mol %) BnEt₃N⁺Cl⁻ (5 mol %), and degassed toluene (10 mL) and water (10 mL). The reaction mixture was refluxed for 16 h to 18 h, and was subsequently partitioned between 1.5 N hydrochloric acid (10 mL) and toluene (100 mL). The toluene extract was concentrated under reduced pressure, and the resulting residue was purified by silica gel column chromatography.

TABLE 2
Preparation of 2,3-bisaryl-2-buten-4-olide (Formula 13) via coupling of
2,3-dichloro-2-buten-4-olide and m-substituted phenylboronic acid
(Formula 12)

| | R | |
|---|---|---|
| Example | Formula 12, 13 | Yield^{a} |
| 12 | H | 93 |
| 13 | CH₃ | 87 |
| 14 | MeO | 94 |
| 15 | Cl | 89 |
| 16 | F | 89 |
| 17 | CF₃ | 99 |

^{a}Isolated yields after chromatography

### Example 12

2,3-Diphenyl-2-buten-4-olide. Pale yellow solid: ¹H NMR (CDCl₃): δ 7.29-7.45 (m, 10H), 5.19 (s, 2H). ¹³C NMR (CDCl₃): δ 173.7, 156.4, 130.9, 130.8, 130.4, 129.4, 129.2, 129.0, 128.8, 127.7, 126.2, 70.8. Anal. Calc'd for C₁₆H₁₂O₂: C, 81.34; H, 5.12. Found: C, 81.08; H, 4.97.

### Example 13

2,3-Di-m-tolyl-2-buten-4-olide. Pale yellow solid: ¹H NMR (CDCl₃): δ 7.09-7.27 (m, 8H), 5.16 (s, 2H), 2.34 (s, 3H), 2.29 (s, 3H). ¹³C NMR (CDCl₃): δ 173.9, 156.3, 138.9, 138.6, 131.6, 131.0, 130.4, 130.0, 129.8, 129.0, 128.7, 128.1, 126.6, 126.3, 125.0, 70.9, 21.7, 21.6. Anal. Calc'd for C₁₈H₁₆O₂: C, 81.79; H, 6.10. Found: 81.75; H, 6.18.

### Example 14

2,3-Bis-(3-methoxy-phenyl)-2-buten-4-olide. Pale yellow gum: ¹H NMR (CDCl₃): δ 7.24-7.31 (m, 2H), 6.83-7.01 (m, 6H), 5.16 (s, 2H), 3.75 (s, 3H), 3.64 (s, 3H). ¹³C NMR (CDCl₃): δ 173.55, 159.9, 156.4, 132.1, 131.7, 130.3, 130.0, 126.4, 121.9, 120.0, 116.8, 115.0, 114.7, 113.1, 70.8, 55.5, 55.4. Anal. Calc'd for C₁₈H₁₆O₄: C, 72.96; H, 5.44. Found: C, 72.71; H, 5.27.

### Example 15

2,3-Bis-(3-chloro-phenyl)-2-buten-4-olide. Pale yellow gum: ¹H NMR (CDCl₃): δ 7.16-7.43 (m, 8H), 5.14 (s, 2H). ¹³C NMR (CDCl₃): δ 172.7, 155.9, 135.3, 134.8, 132.3, 131.5, 131.0, 130.7, 130.2, 129.5, 129.3, 127.6, 127.5, 126.1, 126.0, 70.7. Anal. Calc'd for C₁₆H₁₀Cl₂O₂: C, 62.98; H, 3.30; Cl, 23.24. Found: C, 62.90; H, 3.06; Cl, 23.27.

### Example 16

2,3-Bis-(3-fluoro-phenyl)-2-buten-4-olide. Off-white solid: ¹H NMR (CDCl₃): δ 7.00-7.39 (m, 8H), 5.16 (s, 2H). ¹³C NMR (CDCl₃): δ 172.8, 164.3, 161.8, 155.9, 132.7, 132.6, 131.9, 131.8, 131.3, 131.2, 130.7, 130.6, 126.3, 125.3, 123.6, 118.2, 118.0, 116.6, 116.5, 116.4, 116.3, 114.9, 114.6, 70.8. Anal. Calc'd for C₁₆H₁₀F₂O₂: C, 70.59; H, 3.70; F, 13.96. Found: C, 70.37; H, 3.55; F, 14.13.

### Example 17

2,3-Bis-(3-trifluoromethyl-phenyl)-2-buten-4-olide. Pale yellow gum: ¹H NMR (CDCl₃): δ 7.62-7.71 (m, 4H), 7.46-7.56 (m, 4H), 5.24 (s, 2H). ³C NMR (CDCl₃): δ 171.6, 155.9, 132.9, 131.4, 131.0, 130.5, 130.2, 129.8, 127.9, 126.3, 124.5, 70.7. Anal. Calc'd for C₁₈H₁₀F₆O₂: C, 58.08; H, 2.71; F, 30.62. Found: C, 57.78; H, 2.72; F, 30.91.

### Example 18

### Preparation of 5-hydroxy-3,4-diphenyl-5H-furan-2-one

Mucochloric acid (2.5 mmol) was combined with phenylboronic acid (3.0 equiv.), cesium fluoride (4.0 equiv.), PdCl₂(PPh₃)₂ (5 mol %), BnEt₃N⁺Cl⁻ (5 mol %), and degassed toluene (10 mL) and water (10 mL). The reaction mixture was refluxed for 4 h, and was subsequently quenched with saturated aqueous ammonium chloride solution (100 mL) and extracted with ethyl acetate (2×100 mL). The ethyl acetate extract was washed with saturated aqueous ammonium chloride solution (100 mL) and concentrated under reduced pressure. The residue was purified by silica gel column chromatography to yield an off-white solid. Yield: 71%. ¹H NMR (CDCl₃): δ 7.24-7.44 (m, 10H), 6.50 (d, J=7.6 Hz, 1H), 4.82 (d, J=8.1 Hz, 1H). ¹³C NMR (CDCl₃): δ 171.6, 155.5, 130.8, 130.4, 129.6, 129.4, 129.1, 128.9, 128.4, 97.5. Anal. Calc'd for C₁₆H₁₂O₃: C, 76.18; H, 4.79. Found: C, 75.81; H, 4.76.

### Example 19

### Preparation of 4-benzyloxy-2,3-dichloro-2-buten-4-olide

A mixture of mucochloric acid (16.8 g, 0.100 mol), benzyl alcohol (16.2 g, 1.5 equiv.), AMBERLYST® 15 (0.33 g), zinc chloride (1.4 g, 0.1 equiv.), and toluene (300 mL) was refluxed for 6 h with a Dean-Stark trap in place. The reaction mixture was filtered and the toluene was removed under reduced pressure. The residue was purified by silica gel column chromatography to give 4-benzyloxy-2,3-dichloro-2-buten-4-olide as a pale yellow oil: yield: 13.6 g (77%). ¹H NMR (CDCl₃): δ 7.34-7.43 (m, 5H), 5.85 (s, 1H), 4.92 (d, J=11.5, 1H), 4.77 (d, J=11.5, 1H). ¹³C NMR (CDCl₃): δ 163.5, 147.9, 135.2, 129.0, 128.6, 124.5, 99.8, 71.9. Anal. Calc'd for C₁₁H₈Cl₂O₃: C, 50.99; H, 3.11; Cl, 27.37. Found: C, 51.10; H, 3.05; Cl, 27.42.

### Example 19

### Preparation of 4-benzyloxy-2,3-diphenyl-2-buten-4-olide

4-Benzyloxy-2,3-dichloro-2-buten-4-olide (2.5 mmol) was combined with phenylboronic acid (3.0 equiv.), cesium fluoride (4.0 equiv.), PdCl₂(PPh₃)₂ (5 mol %), BnEt₃N⁺Cl⁻ (5 mol %), and degassed toluene (10 mL) and water (10 mL). The reaction mixture was refluxed for 4 h, and was subsequently quenched with 1.5 N hydrochloric acid (100 mL) and extracted with ethyl toluene (2×100 mL). The toluene extract was washed with water and concentrated under reduced pressure. The residue was purified by silica gel column chromatography to yield 4-benzyloxy-2,3-diphenyl-2-buten-4-olide as an off-white solid. Yield: 90%. ¹H NMR (CDCl₃): δ 7.24-7.45 (m, 15H), 6.29 (s, 1H), 4.94 (d, J=11.5, 1H), 4.84 (d, J=11.5, 1H). ¹³C NMR (CDCl₃): δ 170.8, 153.8, 136.0, 130.7, 130.4, 129.7, 129.6, 129.4, 129.1, 128.9, 128.85, 128.8, 128.7, 100.4, 71.5. Anal. Calc'd for C₂₃H₁₈O₃: C, 80.68; H, 5.30. Found: C, 80.63; H, 5.29.

### Example 20

### Preparation of 2-bromo-3-(4-methylsulfanyl-phenyl)-2-buten-4-olide

2,3-dibromo-2-buten-4-olide (2.3 mmol) was combined with 4-(methylsulfanyl)phenylboronic acid (2.0 equiv.), cesium fluoride (2.67 equiv.), PdCl₂(PPh₃)₂ (5 mol %), BnEt₃N⁺Cl⁻ (5 mol %), and degassed toluene (10 mL) and water (10 mL). The reaction mixture was stirred at 20° C. to 25° C. for 3 d, and was subsequently partitioned between 2 N hydrochloric acid (10 mL) and toluene (100 mL). The toluene extract was concentrated under reduced pressure, and the residue was purified by silica gel column chromatography to yield 2-bromo-3-(4-methylsulfanyl-phenyl)-2-buten-4-olide as a white solid. Yield: 91%; mp 138° C. to 140° C. ¹H NMR (CDCl₃): δ 7.77-7.79 (m, 2H), 7.31-7.33 (m, 2H), 5.17 (s, 2H), 2.53 (s, 3H). ¹³C NMR (CDCl₃): δ 170.1, 155.3, 144.9, 127.69 125.9, 125.4, 105.5, 71.8, 15.1. Anal. Calc'd for C₁₁H₉Br₁O₂S₁: C, 46.33; H, 3.18; Br, 28.02; S, 11.24. Found: C, 46.13; H, 3.19; Br, 28.32; S, 11.04.

### 21

### Preparation of 2-chloro-3-(4-methylsulfanyl-phenyl)-2-buten-4-olide

2,3-Dichloro-2-buten-4-olide (2.3 mmol) was combined with 4-(methylsulfanyl)phenylboronic acid (2.0 equiv.), cesium fluoride (2.67 equiv.), PdCl₂(PPh₃)₂ (5 mol %), BnEt₃N⁺Cl⁻ (5 mol %), and degassed toluene (10 mL) and water (10 mL). The reaction mixture was stirred at 20° C. to 25° C. for 3 d, and was subsequently partitioned between 2 N hydrochloric acid (10 mL) and toluene (100 mL). The toluene extract was concentrated under reduced pressure, and the residue was purified by silica gel column chromatography to give 2-chloro-3-(4-methylsulfanyl-phenyl)-2-buten-4-olide as a white solid. Yield: 99%; mp 156° C. to 158° C. ¹H NMR (CDCl₃): δ 7.70-7.73 (m, 2H), 7.29-7.33 (m, 2H), 5.19 (s, 2H), 2.52 (s, 3H). ¹³C NMR (CDCl₃): δ 169.5, 151.4, 144.8, 127.6, 125.9, 124.9, 116.3, 70.2, 15.0. Anal. Calc'd for C₁₁H₉Cl₁O₂S₁: C, 54.89; H, 3.77; Cl, 14.73; S. 13.32. Found: C, 54.93; H, 3.80; Cl, 14.48; S, 13.24.

### Example 22

### Preparation of 3-(4-methylsulfanyl-phenyl)-2-phenyl-2-buten-4-olide

2-Bromo-3-(4-methylsulfanyl-phenyl)-2-buten-4-olide (1.0 mmol) was combined with phenylboronic acid (2.0 equiv.), cesium fluoride (3.0 equiv.), PdCl₂(PPh₃)₂ (5 mol %), BnEt₃N⁺Cl⁻ (5 mol %), and degassed toluene (10 mL) and water (10 mL). The reaction mixture was stirred at 20° C. to 25° C. for 2-3 d, and was subsequently partitioned between 2 N hydrochloric acid (10 mL) and toluene (100 mL). The toluene extract was concentrated under reduced pressure, and the residue was purified by silica gel column chromatography to yield 3-(4-methylsulfanyl-phenyl)-2-phenyl-2-buten-4-olide as a white solid. Yield: 95%. ¹H NMR (CDCl₃): δ 7.37-7.44 (m, 5H), 7.22-7.25 (m, 2H), 7.14-7.17 (m, 2H), 5.16 (s, 2H), 2.48 (s, 3H), ¹³C NMR (CDCl₃): δ 173.8, 155.6, 143.0, 130.6, 129.5, 129.0, 128.0, 127.1, 126.0, 125.6, 70.6, 15.1. Anal. Calc'd for C₁₇H₁₄O₂S₁: C, 72.31; H, 5.00; S, 11.36. Found: C, 72.01; H, 4.87.

### Example 23

### Preparation of 2-chloro-3-(4-methanesulfonyl-phenyl)-2-buten-4-olide

2-Chloro-3-(4-methylsulfanyl-phenyl)-2-buten-4-olide (1.4 mmol) was combined with OXONE® (potassium peroxymonosulfate, 3.0 equiv.), acetone (20 mL) and water (600 μL). The reaction mixture was stirred at 0-25° C. for 24 h. The reaction mixture was filtered and the solids washed with acetone. The acetone solution was concentrated under reduced pressure, and the residue was washed with water to yield 2-chloro-3-(4-methanesulfonyl-phenyl)-2-buten-4-olide as a white solid. No further purification was necessary. Yield: 85%. ¹H NMR (CDCl₃): δ 8.10 (d, J=8.5, 2H), 7.98 (d, J=8.5, 2H), 5.25 (s, 2H), 3.10 (s, 3H). ¹³C NMR (DMSO-d₆): δ 169.1, 153.1, 143.4, 133.8, 129.2, 128.3, 118.3, 71.5, 43.9. Anal. Calc'd for C₁₁H₉Cl₁O₄S₁: C, 48.45; H, 3.33; Cl, 13.00. Found: C, 48.42; H, 3.15; Cl, 12.66.

### Example 24

### Preparation of 3-(4-methanesulfonylphenyl)-2-phenyl-2-buten-4-olide (rofecoxib)

2-Chloro-3-(4-methanesulfonyl-phenyl)-2-buten-4-olide (0.77 mmol) was combined with phenylboronic acid (2.0 equiv.), cesium fluoride (2.0 equiv.), PdCl₂(PPh₃)₂ (5 mol %), BnEt₃N⁺Cl⁻ (5 mol %), and degassed toluene (10 mL) and water (10 mL). The reaction mixture was refluxed for 7 h, and was subsequently partitioned between 2 N hydrochloric acid (10 mL) and toluene (100 mL). The toluene extract was concentrated under reduced pressure, and the resulting residue was purified by silica gel column chromatography to yield 3-(4-methanesulfonyl-phenyl)-2-phenyl-2-buten-4-olide as a white solid. Yield: 74%; mp 197° C. to 199° C. (dec.). ¹H NMR (CDCl₃): δ 7.92 (d, J=8.5, 2H), 7.51 (d, J=8.5, 2H), 7.38-7.42 (m, 5H), 5.20 (s, 2H), 3.07 (s, 3H). ¹³C NMR (DMSO-d₆): δ 173.2, 156.7, 142.6, 136.3, 130.4, 129.8, 129.6, 129.4, 129.3, 128.1, 127.5, 124.9, 71.5, 43.8. Anal. Calc'd for C₁₇H₁₄O₄S₁: C, 64.95; H, 4.49. Found: C, 64.65; H, 4.40.

### Example 25

### Preparation of 3-(4-methanesulfonylphenyl)-2-phenyl-2-buten-4-olide (rofecoxib)

3-(4-Methylsulfanyl-phenyl)-2-phenyl-2-buten-4-olide (0.71 mmol) was combined with OXONE® (potassium peroxymonosulfate, 3.0 equiv.), acetone (10 mL) and water (300 μL). The reaction mixture was stirred at 0-25° C. for 24 h. The reaction mixture was filtered and the solids washed with acetone. The acetone solution was concentrated under reduced pressure, and the residue was washed with water to yield 3-(4-methanesulfonylphenyl)-2-phenyl-2-buten-4-olide as a white solid. Yield: 95%. No further purification was necessary.

### Example 26

### Preparation of 4-benzyloxy-2,3-dibromo-2-buten-4-olide

A mixture of mucochloric acid (25.8 g, 0.100 mol), benzyl alcohol (16.2 g, 1.5 equiv.), p-toluenesulfonic acid monohydrate (0.958 g), and toluene (400 mL) was refluxed for 24 h with a Dean-Stark trap in place to remove water. The reaction mixture was cooled to RT and filtered. The toluene was removed under reduced pressure, and the resulting residue was purified by silica gel column chromatography to give 4-benzyloxy-2,3-dibromo-2-buten-4-olide: yield: 33.2 g (95%). ¹H NMR, ¹³C NMR, MS, and elemental analysis were consistent with the titled compound.

### Example 27

### Preparation of 2,3-bis-(1-benzenesulfonyl-1H-indol-3-yl)-4-benzyloxy-2-buten-4-olide

4-Benzyloxy-2,3-dibromo-2-buten-4-olide (1.1 g, 2.9 mmol) was combined with an 1-benzenesulfonyl-1H-indol-3-yl-boronic acid (2.04 g, 2.2 equiv.), cesium fluoride (4.4 equiv.), PdCl₂(PPh₃)₂ (5 mol %), BnEt₃N⁺Cl⁻ (5 mol %), and degassed toluene (20 mL) and water (20 mL). The reaction mixture was refluxed for 20 h, cooled to RT, quenched with EtOAc (100 mL), and washed with aq NaCl (150 mL). The toluene-rich phase was concentrated under reduced pressure, and the resulting residue was purified by silica gel column chromatography to give 2,3-bis-(1-benzenesulfonyl-1H-indol-3-yl)-4-benzyloxy-2-buten-4-olide as a yellow solid (yield: 29%), and a corresponding mono-indolyl product as a white solid (yield: 39%).

It should be noted that, as used in this specification and the appended claims, singular articles such as “a,” “an,” and “the,” may refer to a single object or to a plurality of objects unless the context clearly indicates otherwise. Thus, for example, reference to a composition containing “a compound” may include a single compound or two or more compounds.

It is to be understood that the above description is intended to be illustrative and not restrictive. Many embodiments will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications, granted patents, and publications, are incorporated herein by reference in their entirety and for all purposes.

1. A method of making a compound of Formula 1, 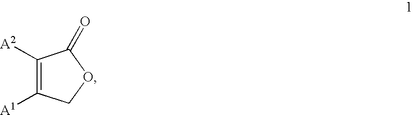
wherein Ar¹ and Ar² are the same or different, and are aryl, the method comprising:
(a) reacting a compound of Formula 2, 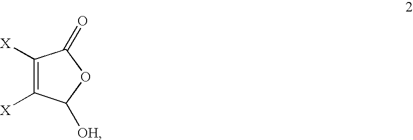
with a reducing agent, in the presence of an acid catalyst and a first solvent to yield a compound of Formula 3, 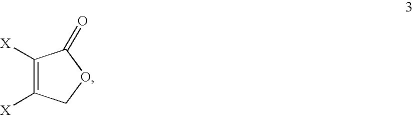
wherein X in Formula 2 and in Formula 3 is halogen;
(b) reacting the compound of Formula 3 with a compound of Formula 4,
Ar¹—B(OH)₂ 4,
at temperature T₁, and in the presence of a first base, a first Pd catalyst, a first phase transfer catalyst, a second solvent, and H₂O, to yield the compound of Formula 5, 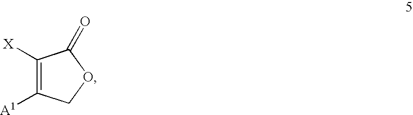
wherein Ar¹ in Formula 4 and Formula 5 are as defined above for Formula 1, X is as defined above for Formula 2 and Formula 3, and the second solvent is the same as or different than the first solvent; and
(c) reacting the compound of Formula 5 with a compound of Formula 6,
Ar²—B(OH)₂ 6,
at temperature T₂, and in the presence of a second base, a second Pd catalyst, a second phase transfer catalyst, a third solvent, and H₂0, to yield the compound of Formula 1;
wherein Ar2 in Formula 6 is as defined above in Formula 1;
the second base, second Pd catalyst, second phase transfer catalyst, and third solvent are the same as or different than, the first base, the first Pd catalyst, the first phase transfer catalyst, and the second solvent, respectively; and
T₂ is greater than T₁.

2. The method of claim 1, wherein T₂ is about reflux temperature or below.

3. The method of claim 1, wherein T₁ is about room temperature or below.

4. A method of making a compound of Formula 7, 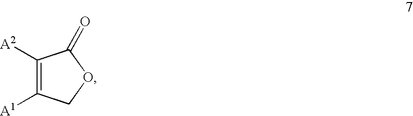
wherein Ar¹ is aryl, the method comprising:
(a) reacting a compound of Formula 2, 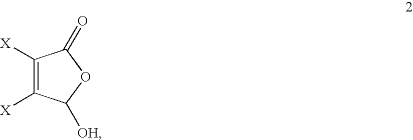
with a reducing agent, in the presence of an acid catalyst and a first solvent to yield a compound of Formula 3, 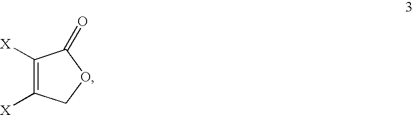
wherein X in Formula 2 and Formula 3 is halogen;
(b) reacting the compound of Formula 3 with a compound of Formula 4,
Ar¹—B(OH)₂ 4,
at a temperature above room temperature, and in the presence of a base, a Pd catalyst, a phase transfer catalyst, a second solvent, and H₂O, to yield the compound of Formula 7;
wherein Ar¹ in Formula 4 is as defined above for Formula 7, X is as defined above for Formula 2 and Formula 3, and the second solvent is the same as or different than the first solvent.

5. The method of claim 3, wherein the compound of Formula 3 is reacted with the compound of Formula 4 at about reflux temperature or below.

6. A method of making a compound of Formula 5, 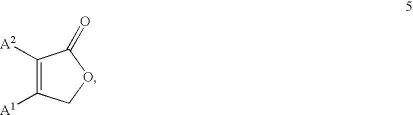
wherein Ar¹ is aryl, and X is halogen, the method comprising:
(a) reacting a compound of Formula 2, 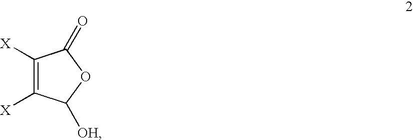
with a reducing agent, in the presence of an acid catalyst and a first solvent to yield a compound of Formula 3, 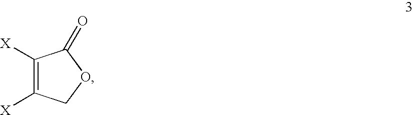
wherein X in Formula 2 and in Formula 3 is as defined above in Formula 5;
(b) reacting the compound of Formula 3 with a compound of Formula 4,
Ar¹—B(OH)₂ 4,
at a temperature below reflux temperature, and in the presence of a base, a Pd catalyst, a phase transfer catalyst, a second solvent, and H₂O, to yield the compound of Formula 5;
wherein Ar¹ in Formula 4 is as defined above for Formula 5, and the second solvent is the same as or different than the first solvent.

7. The method of claim 6, wherein the compound of Formula 3 is reacted with the compound of Formula 4 at about room temperature or below.
